(19) United States
(12) Patent Application Publication
(10) Pub. No.: US 2026/0223860 A1
(43) Pub. Date: Aug. 6, 2026

(54) BACTERIOPHAGES FOR THE CONTROL OF BACTERIAL SPOT AND BACTERIAL SPECK DISEASE

(71) Applicant: ECOPHAGE LTD., Nes Ziona (IL)

(72) Inventors: Guy ELITZUR, RaAnana (IL); Sammy FRENK, Kfar Saba (IL); Yuval Efraim WINER, Givataim (IL); Itay KEREN, Kibbutz GvarAm (IL); Shiri TOPMAN RAKOVER, Rehovot (IL)

(21) Appl. No.: 19/147,090

(22) PCT Filed: Feb. 15, 2024
(86) PCT No.: PCT/IL2024/050175

§ 371 Date: Jul. 10, 2025

Related U.S. Application Data
(60) Provisional application No. 63/446,066, filed on Feb. 16, 2023.

(51) Int. Cl. A01N 63/40; A01P 1/00; C12N 7/00

(52) U.S. Cl. CPC A01N 63/40; A01P 1/00; C12N 7/00; C12N 2795/00031; C12N 2795/00071

(57) ABSTRACT

Agricultural compositions are disclosed which comprise at least one isolated bacteriophage capable of infecting a plant pathogen of the genus Xanthomonas and, optionally, at least one isolated bacteriophage strain capable of infection the plant pathogen Pseudomonas syringae pv. tomato. Uses thereof for treating bacterial spot and bacterial speck disease are also disclosed.

### FIELD OF THE INVENTION

The present invention relates to agricultural compositions comprising bacteriophages for the control of bacterial plant pathogens. More particularly, the invention relates to compositions comprising at least one isolated bacteriophage strain capable of infecting the plant pathogen of the genus Xanthomonas and, optionally, at least one isolated bacteriophage strain capable of infection the plant pathogen Pseudomonas syringae pv. tomato. Uses thereof for treating bacterial spot or bacterial speck disease are also disclosed.

### BACKGROUND OF THE INVENTION

Bacteriophages are viruses that specifically infect bacteria. The emergence of bacterial resistance to antibiotics and copper-based bactericides in recent years resulted in an increasing interest in bacteriophage therapy for the treatment of human and livestock infections, as well as crop protection.

Within the crop protection disciplines, the prevention and treatment of bacterial diseases remain an unresolved challenge with limited available solutions. Damage caused by bacterial pathogens results in a considerable loss of yield, which in turn costs farmers billions of dollars in annual lost revenues. Common agricultural practices for treating bacterial infections include the application of chemical-based compounds, such as the broad-spectrum antibiotic streptomycin, or copper-based bactericides. However, in recent years the efficacy of these compounds has been reduced in many regions due to the emergence of resistant variants. Furthermore, the toxicity of these compounds to plants and to the environment results in poor public opinion.

One major bacterial disease in tomato and pepper plants is bacterial spot. This disease is caused by four Xanthomonas species: Xanthomonas vesicatoria, Xanthomonas perforans, Xanthomonas gardneri, and Xanthomonas euvesicatoria (i.e. Xanthomonas campestris pv. vesicatoria). Another disease is the bacterial speck disease in tomato plants that is caused by Pseudomonas syringae pv. tomato. Altogether, these diseases can lead to yield losses of up to 60% and cause annual damages estimated in billions of dollars. Bacteria can spread from plant to plant by tools, workers' hands, or through splashing rain or irrigation water. Therefore, a preventative treatment, where the active ingredient (e.g., bacteriophage) is applied prophylactically can be especially useful in preventing and reducing the disease symptoms.

### SUMMARY OF THE INVENTION

In one aspect of the invention, there is provided an agricultural composition comprising:
(i) at least one isolated bacteriophage capable of infecting a plant pathogen of the genus Xanthomonas, said at least one bacteriophage having a genomic nucleic acid sequence at least 85% identical to the nucleic acid sequence as set forth in SEQ ID NOs: 5, 6, 7, 8, or 9; and
(ii) an agriculturally acceptable carrier,
wherein the composition comprises no more than 10 different strains of bacteriophage.

According to one embodiment of the invention, the composition comprises at least two bacteriophages capable of infecting the pathogen of the genus Xanthomonas

According to a specific embodiment of the invention, the agricultural composition comprises:
(i) a first bacteriophage having a genomic nucleic acid sequence at least 85% identical to the nucleic acid sequence as set forth in SEQ ID NO: 5; and
(ii) a second bacteriophage having a genomic nucleic acid sequence at least 85% identical to the nucleic acid sequence as set forth in SEQ ID NO: 6.

According to another specific embodiment of the invention, the agricultural composition comprises:
(i) a first bacteriophage having a genomic nucleic acid sequence at least 85% identical to the nucleic acid sequence as set forth in SEQ ID NO: 5; and
(ii) a second bacteriophage having a genomic nucleic acid sequence at least 99% identical to the nucleic acid sequence as set forth in SEQ ID NO: 9.

In some embodiments of the invention, the agricultural composition further comprises at least one additional isolated bacteriophage capable of infecting a plant pathogen of the species Pseudomonas syringae pv. tomato.

According to one embodiment of the invention, said at least one additional bacteriophage has a genomic nucleic acid sequence at least 85% identical to the nucleic acid sequence as set forth in SEQ ID NOs: 1, 2, 3, or 4.

According to a further embodiment of the invention, the agricultural composition comprises no additional bacteriophage that infects plant pathogens other than Pseudomonas syringae pv. tomato or pathogens of the genus Xanthomonas.

According to yet another embodiment of the invention, said at least one bacteriophage capable of infecting Pseudomonas syringae pv. tomato comprises at least three non-identical bacteriophages capable of infecting Pseudomonas syringae pv. tomato.

According to a specific embodiment of the invention, said at least three non-identical bacteriophages capable of infecting Pseudomonas syringae pv. tomato comprise:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2; and
(iii) a bacteriophage having a genomic nucleic acid sequence at least 85% identical to the nucleic acid sequence as set forth in SEQ ID NO: 3.

According to another specific embodiment of the invention, said at least three non-identical bacteriophages capable of infecting Pseudomonas syringae pv. tomato comprise:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2; and
(iii) a bacteriophage having a genomic nucleic acid sequence at least 85% identical to the nucleic acid sequence as set forth in SEQ ID NO: 4.

In another aspect of the invention, there is provided an agricultural composition comprising:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 5; and
(iv) a bacteriophage having a genomic nucleic acid sequence at least 85% identical to the nucleic acid sequence as set forth in SEQ ID NO: 6;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 34;
(v) a bacteriophage having a genomic nucleic acid sequence at least 85% identical to the nucleic acid sequence as set forth in SEQ ID NO: 5;
(vi) a bacteriophage having a genomic nucleic acid sequence at least 85% identical to the nucleic acid sequence as set forth in SEQ ID NO: 7;
(vii) a bacteriophage having a genomic nucleic acid sequence at least 85% identical to the nucleic acid sequence as set forth in SEQ ID NO: 8; and
(viii) a bacteriophage having a genomic nucleic acid sequence at least 85% identical to the nucleic acid sequence as set forth in SEQ ID NO: 9.

According to a particular embodiment of the invention, the at least one isolated bacteriophage capable of infecting a plant pathogen of the genus Xanthomonas is a bacteriophage deposited under accession number F/00201, F/00202, F/00203, or I-6020.

According to another particular embodiment of the invention, the at least one isolated bacteriophage capable of infecting a plant pathogen of the species Pseudomonas syringae pv. tomato is a bacteriophage deposited under accession number F/00196, F/00197, F/00198, or F/00200.

According to yet another embodiment of the invention, the composition further comprises an agent that promotes the growth of a plant or maintains the health of the plant.

In a specific embodiment of the invention, said agent is selected from the group consisting of a fertilizer, an acaricide, a herbicide, a fungicide, an insecticide, a nematicide, a pesticide, a plant growth regulator, a rodenticide, and a nutrient.

According to a further embodiment of the invention, said agriculturally acceptable carrier comprises at least one agent selected from the group consisting of a stabilizer, a tackifier, a preservative, a surfactant, an adjuvant, a salt, and any combination thereof.

In yet a further aspect of the invention, there is provided an article of manufacture comprising:
(i) at least one bacteriophage capable of infecting a plant pathogen Pseudomonas syringae pv. Tomato, the bacteriophage having a genomic nucleic acid sequence at least 85% identical to the nucleic acid sequence as set forth in SEQ ID NOs: 1, 2, 3, or 4; and
(ii) at least one bacteriophage capable of infecting a plant pathogen of the genus Xanthomonas having a genomic nucleic acid sequence at least 85% identical to the nucleic acid sequence as set forth in SEQ ID NOs: 5, 6, 7, 8, or 9.

In some embodiments, wherein said at least one bacteriophage capable of infecting the pathogen Pseudomonas syringae pv. tomato and said at least one bacteriophage capable of infecting the pathogen of the genus Xanthomonas are formulated in a single composition.

According to one embodiment of the invention, said composition comprises no more than 10 bacteriophages capable of infecting pathogens of the species Pseudomonas syringae pv. tomato and pathogens of the genus Xanthomonas.

In other embodiments, said at least one bacteriophage capable of infecting the pathogen Pseudomonas syringae pv. tomato and said at least one bacteriophage capable of infecting the pathogen of the genus Xanthomonas are formulated in separate compositions.

According to one embodiment of the invention, each of said separate compositions comprises no more than 10 phages capable of infecting pathogens of the species Pseudomonas syringae pv. tomato and pathogens of the genus Xanthomonas.

In a specific embodiment of the invention, the article of manufacture comprises:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 5; and
(iv) a bacteriophage having a genomic nucleic acid sequence at least 85% identical to the nucleic acid sequence as set forth in SEQ ID NO: 6;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 4;
(v) a bacteriophage having a genomic nucleic acid sequence at least 85% identical to the nucleic acid sequence as set forth in SEQ ID NO: 5;
(vi) a bacteriophage having a genomic nucleic acid sequence at least 85% identical to the nucleic acid sequence as set forth in SEQ ID NO: 7;
(vii) a bacteriophage having a genomic nucleic acid sequence at least 85% identical to the nucleic acid sequence as set forth in SEQ ID NO: 8; and
(viii) a bacteriophage having a genomic nucleic acid sequence at least 85% identical to the nucleic acid sequence as set forth in SEQ ID NO: 9.

According to a further specific embodiment of the invention, the at least one isolated bacteriophage capable of infecting a plant pathogen of the genus Xanthomonas is a bacteriophage deposited under accession number F/00201, F/00202, F/00203, or I-6020.

According to yet a further embodiment of the invention, the at least one isolated bacteriophage capable of infecting a plant pathogen of the species Pseudomonas syringae pv. tomato is a bacteriophage deposited under accession number F/00196, F/00197, F/00198, or F/00200.

In another aspect, the present invention provides a method of controlling or preventing bacterial spot disease of a tomato or pepper plant comprising contacting the tomato or pepper plant with an effective amount of at least one bacteriophage capable of infecting a pathogen of the genus Xanthomonas, said at least one bacteriophage having a genomic nucleic acid sequence at least 85% identical to the nucleic acid sequence as set forth in SEQ ID NOs 5, 6, 7, 8, or 9, thereby controlling or preventing the bacterial spot disease of the tomato or pepper plant.

According to one embodiment of the invention, the at least one bacteriophage capable of infecting a pathogen of the genus Xanthomonas, is a bacteriophage having a genomic nucleic acid sequence at least 85% identical to the nucleic acid sequence as set forth in SEQ ID NOs: 5 or 6, or a bacteriophage having a genomic nucleic acid sequence at least 99% identical to the nucleic acid sequence as set forth in SEQ ID NOs: 7, 8 or 9.

In yet another aspect of the invention, there is provided a method of controlling or preventing bacterial spot disease and bacterial speck disease of a tomato plant comprising contacting the tomato plant with an effective amount of the agricultural composition of any one of claims 5-16, or the article of manufacture of any one of claims 22-34, thereby controlling or preventing the bacterial spot disease and bacterial speck disease of the tomato plant.

According to one embodiment of the invention, said contacting is carried out in the vicinity of or onto: a shoot, root, stalk, stem, trunk, seed, fruit, flower, leaf, or any combination thereof.

According to another embodiment of the invention, said contacting comprises dipping, spraying, irrigating, drenching, or fogging.

According to yet another embodiment of the invention, the contacting is repeated daily, weekly, twice a week, every other week, or monthly.

### BRIEF DESCRIPTION OF THE DRAWINGS

FIG. 1 shows the efficacy of treatment of tomato plants affected by Xanthomonas campestris p.v. vesicatoria (Xcv) and Pseudomonas syringae p.v. tomato (Pst) with a combination of bacteriophages according to one embodiment of the invention or treatment with Cupravit® Hydro compared to untreated control. In this FIG.: BP (bacteriophage cocktail treatment), C (Cupravit® Hydro treatment), D (days), E (efficacy).

FIG. 2 shows the chlorophyll content, corresponding to SPAD value, in leaves of tomato plants affected by Xanthomonas campestris p.v. vesicatoria (Xcv) and Pseudomonas syringae p.v. tomato (Pst) treated with a combination of bacteriophages according to one embodiment of the invention or with Cupravit® Hydro compared to untreated control. In this FIG.: BP (bacteriophage cocktail treatment), C (Cupravit® Hydro treatment), UTC (untreated control).

FIG. 3 shows images of leaves on the day of the first application (D0) and 7 days after the onset of treatment (D7) of tomato plants tomato plants affected by Xanthomonas campestris p.v. vesicatoria (Xcv) and Pseudomonas syringae p.v. tomato (Pst) treated with a combination of bacteriophages according to one embodiment of the invention or with Cupravit® Hydro compared to untreated control. In this FIG.: BP (bacteriophage cocktail treatment), C (Cupravit® Hydro treatment), S (symptoms of spot and/or speck disease manifested as dark lesions) UTC (untreated control).

### DETAILED DESCRIPTION OF THE INVENTION

The present invention relates to agricultural compositions comprising bacteriophages for the control of plant pathogens.

It should be noted that the present invention may be carried out in various ways and shall not be limited by the following description and examples.

Plant diseases caused by bacterial pathogens are vast and include systemic diseases and local infections. Two major diseases in tomato, with similar symptoms, are bacterial spot, which also affects pepper plants, and bacterial speck, both manifested by black spots on foliage and on fruit. Bacterial spot is caused by four species of the genus Xanthomonas: Xanthomonas vesicatoria, Xanthomonas perforans, Xanthomonas gardneri, and Xanthomonas euvesicatoria (i.e. Xanthomonas campestris pv. vesicatoria). Bacterial speck disease in tomato is caused by Pseudomonas syringae pv. tomato. Altogether, these diseases can lead to yield losses of up to 60% and cause annual damages estimated in billions of dollars. Conventional treatments, many of them are based on copper substances, are considered as environmental pollutants and their effectiveness is declining due to the increasing prevalence of tolerant strains. Bacteria can spread in the field from plant to plant by tools, workers' hands, or through splashing rain or irrigation water.

The present inventors have uncovered novel bacteriophages that are capable of infecting, replicating within, and lysing Xanthomonas species. These bacteriophages are thus useful in controlling the bacterial spot.

The present inventors also revealed that particular combinations of these novel phages exhibit synergistic ability to lyse the bacteria and, therefore, bring about an enhanced ability to control bacterial spot in plants.

According to one aspect of the present invention, a method for controlling or preventing bacterial spot disease in tomato and/or pepper plants is provided, the method comprising contacting a tomato and/or pepper plant with an effective amount of at least one bacteriophage capable of infecting the pathogen of the genus Xanthomonas, the at least one bacteriophage having a genomic nucleic acid sequence at least 85% identical to the nucleic acid sequence as set forth in SEQ ID NOs: 5 or 6, or at least one bacteriophage having a genomic nucleic acid sequence at least 99% identical to the nucleic acid sequence as set forth in SEQ ID NOs: 7, 8 or 9.

The term “plant” as used herein encompasses whole plants, a grafted plant, ancestors and progeny of the plants, and plant parts, including seeds, shoots, stems, roots, stalks, trunks, tubers, rootstock, scion, foliage, fruits, flowers and plant cells, tissues, and organs. The plant may be in any form including suspension cultures, embryos, meristematic regions, callus tissue, leaves, gametophytes, sporophytes, pollen, and microspores.

In one embodiment, the plant is an agricultural plant including, but not limited to, a tomato plant or a pepper plant. The plants can be of a cultivated genetic background or a wild tomato genetic background.

As used herein, the term “tomato” refers to a plant, line, or population within the species Solanum lycopersicum (synonyms are Lycopersicon lycopersicum or Lycopersicon esculentum) or formerly known under the genus name of Lycopersicon including but not limited to L. cerasiforrne, L. cheesmanii, L. chilense, L. chmielewskii, L. esculentum (now S. pennellii), L. hirsutum, L. parviborum, L. pennellii, L. peruvianum, L. pimpinellifolium, or S. lycopersicoides. The newly proposed scientific name for L. esculentum is S. pennellii. Similarly, the names of the wild species may be altered. L. pennellii has become S. pennellii, L. hirsutum may become S. habrochaites, L. peruvianum may be split into S. ‘N peruvianum’ and S. ‘Callejon de Hueyles’, S. peruvianum, and S. corneliomuelleri, L. parviflorum may become S. neorickii, L. chmielewskii may become S. chmielewskii, L. chilense may become S. chilense, L. cheesmaniae may become S. cheesmaniae or S. galapagense, and L. pimpinellifolium may become S. pimpinellifolium.

Generally, a cultivated tomato refers to a tomato that is suitable for consumption and meets the requirements for commercial cultivation, e.g., typically classified as Solanum lycopersicum. In addition to the parts of tomato plants that are suitable for consumption, such as the fruit, the plant also includes parts thereof or derivatives of the plant that are suitable for propagation. Examples of parts suitable for propagation are organ tissues, such as leaves, stems, roots, shoots, and the like, protoplasts, somatic embryos, anthers, petioles, cells in culture, and the like. Derivatives suitable for propagation are, for instance, seeds. The plants according to the invention can be cultivated or propagated in any conventional manner, as well as by means of tissue culture techniques from plant parts.

The term “tomato” also includes its possible varieties, from marble-sized grape or cherry tomatoes to juicy salad tomatoes, meaty paste tomatoes, and huge, sweet, beefsteak tomatoes, having colors ranging from deep crimson to orange, yellow, green, purple, and chocolate. The availability of a certain variety depends on market demand, regional adaptability, disease resistance, and the end use of the product. Exemplary segments for fresh market tomatoes include, but are not limited to, Beef (fruit weight of about 220-400 gr), Standard (fruit weight of about 160-220 gr), and Cluster (uniform fruit weight of about 120-180 gr). Such varieties are available from major seed companies e.g., Grodena, Macarena, Estatio, Zouk, Climbo, and Climstar, all available from Syngenta. Other varieties can be proprietary or available from other vendors, including but not limited to, Cherry-micro (up to 5 gr) round cherry, mini round cherry (7.5-15 gr), mini plum elongated cherry (10-25 gr). Examples of these varieties are: Creativo (Clause), Batico (Nirit seeds), and Shiren (Hazera Genetics). Cocktail round and elongated (25-40 gr): Romanita, Cherry, and Cocktail with red, yellow, orange, pink, zebra, chocolate background. Examples include, but are not limited to, Summer sun (Hazera Genetics), Black pearl (Burpee) Tyty (Tomodori). Roma determinate and indeterminate. 120-200 gr. Examples of the intermediate marker include, but are not limited to, Lancelot (Vilomorin) and Parsifal (Vilomorin). Pink tomato can be further categorized as beef (220-400), standard (160-220), and cluster (120-180). Example: Momotaro type, Cor di bue tomato, (150-350 gr), Pinton (250-300 gr), open field tomato-determinate or semi-determinate (180-400 gr).

Exemplary cultivars of processing tomatoes include, but are not limited to, Roma, SUN 6366, AB 2, Heinz 9780, Heinz 9557, Halley 3155, and Hypeel 303.

There are two major types of tomato growth: determinate and indeterminate. Determinate growth produces “bush” tomatoes, which are bred for compactness. The entire plant stops growing once the terminal fruit ripens, the remainder of the fruit all ripen nearly simultaneously, and then the plant dies. Indeterminate growth produces tomatoes that can grow up to 10 feet in height (so-called “vining” tomatoes) and will only stop growing when killed (e.g. by frost). Their fruits ripen sequentially. In a typical plant, all growth arises from the reiteration of modular sympodial units that each produce three leaves and a multi-flowered inflorescence. Most field-grown varieties of tomato, including M82, are determinate plants whose shoots produce an average of six sympodial units, each harboring a single inflorescence, within which leaf number gradually decreases before a precocious termination of growth. In general, determinate tomatoes are suitable for open-field production. Semi-determinate and indeterminate “cultivated” varieties are suitable for staked cultivation in the open field or protected nets and for glasshouse cultivation.

According to an embodiment of the invention, the tomato plant is of the determinate type. According to another embodiment of the invention, the tomato plant is of the indeterminate type. According to yet another embodiment of the invention, the tomato plant is of the semi-determinate type.

According to a further embodiment of the invention, the tomato is selected from the group consisting of a single fruit per truss, branched tomato, and cherry tomato.

As used herein, the term “pepper” refers to a plant, line, or population within the species Capsicum annuum from the family Solanaceae (nightshades), within the genus Capsicum. The pepper plant produces berries of many colors (including red, yellow, orange, green, white, chocolate, candy cane, striped, and purple) and has a range of tastes between sweet and spicy hot. The term “pepper” also includes its possible varieties, including Bell pepper, Pimiento (also known as pimento or cherry pepper), Cubanelle, Chili pepper (including banana pepper, which is also known as the yellow wax pepper or banana chili; Thai pepper, which is also known as Thai chili or bird's eye chili; Chiltepin; Ghost pepper; and New Mexico chile), Paprika, Jalapeño, Serrano, Cayenne, Poblano, Habanero, Tabasco, Fish pepper, NuMex Twilight pepper, Chinese Five-Spice pepper, Aji Amarillo, Jimmy Nardello, and Ancho.

It should be noted that while most cultivations of peppers are for culinary purposes, several cultivars within the Capsicum annuum species are bred solely for their aesthetic appeal. These varieties come in various shapes, sizes, and colors, including miniature peppers, variegated foliage, and unusual pod formations, making them popular ornamental plants. These Capsicum annuum ornamentals varieties are also contemplated here.

As used herein, the terms “bacteriophage” and “phage” are used interchangeably and refer to an isolated virus that is capable of infecting a bacterium. Typically, a bacteriophage will be characterized by: 1) the nature of the nucleic acids that make up its genome, e.g., DNA, RNA, single-stranded or double-stranded and/or the genomic sequence thereof; 2) the nature of its infectivity, e.g., lytic or temperate; 3) the receptor on the bacterium to which the bacteriophage is attached during the infection; and 4) the particular bacterial species that it infects (and in certain instances, the particular strain of that species). The bacterial species that can be infected by the phage are termed “host range”.

As used herein, the phrases “isolated bacteriophage”, “isolate”, or grammatical equivalents thereof refer to a bacteriophage that was removed from its natural environment (e.g., removed from the bacteria which the phage typically infects or from the soil, water, and other material in which the phage dwells). In one embodiment of the invention, the isolated bacteriophage is removed from cellular material and/or other elements that naturally exist in the source clinical or environmental sample. The term isolated bacteriophages includes such bacteriophages isolated from the environment (“environmental isolates”).

In one embodiment of the invention, the bacteriophages are lytic. In another embodiment of the invention, the bacteriophages are lysogenic.

The term “lytic bacteriophage” refers to a bacteriophage that infects a bacterial host and causes that host to lyse without incorporating the bacteriophage nucleic acids into the host genome. A lytic bacteriophage is typically not capable of reproducing using the lysogenic cycle. The terms “lysogenic bacteriophage” and “temperate bacteriophage” refer to a bacteriophage that is capable of reproducing using the lysogenic cycle.

As used herein, the phrase “bacteriophage strain” refers to the sequenced phage, as described herein.

The bacterial genus Xanthomonas refers to the gram-negative bacterial pathogen which is the causal agent of bacterial spot disease in tomatoes and peppers.

Exemplary species of the genus Xanthomonas that are infected by the bacteriophage strains of the present invention are those that are found in environmental specimens (e.g., soil, water, and other materials).

According to a specific embodiment of the invention, the bacterial species that are infected by the bacteriophage disclosed herein are Xanthomonas euvesicatoria (i.e. Xanthomonas campestris pv. vesicatoria), Xanthomonas perforans, Xanthomonas gardneri, and Xanthomonas vesicatoria. According to another specific embodiment of the invention, the bacteria species that is infected by the bacteriophages disclosed herein is Xanthomonas campestris pv. vesicatoria.

In some embodiments of the invention, the phages described herein are capable of infecting at least one species of the genus Xanthomonas that is causative for bacterial spot disease.

According to one embodiment of the invention, the isolated bacteriophage that is capable of infecting at least one species of the genus Xanthomonas is a bacteriophage having a genomic nucleic acid as set forth in SEQ ID NOs: 5-9.

Also contemplated herein are progeny of the bacteriophages that have a genomic nucleic acid as set forth in SEQ ID NOs: 5-9 (namely, the parent bacteriophages), wherein the progeny is capable of infecting the same species (or even strain) of bacteria as its parent. Such progeny may have genomes that are identical to the genome of the parent bacteriophage or have genomes comprising a nucleic acid sequence that is at least 50% identical, 60% identical, 70% identical, 80% identical, 85% identical, 90% identical, at least 91% identical, at least 92% identical, at least 93% identical, at least 94% identical, at least 95% identical, 96% identical, 97% identical 98% identical, 99% identical, 99.5% identical, or 99.9% identical to the genome of the parent bacteriophage having the genomic nucleic acid sequence as set forth in SEQ ID NOs: 5 or 6; or at least 85% identical, 90% identical, 95%, identical, 99% identical, 99.1% identical, 99.2% identical, 99.3% identical, 99.4% identical, 99.5% identical, 99.6% identical, 99.7% identical, 99.8% identical, 99.9% identical, or 100% to SEQ ID NOs: 7, 8, or 9.

As used herein, the term “progeny of the bacteriophage” refers to bacteriophages stemming from or derived from the strains identified herein.

Also contemplated herein are functional homologs of those bacteriophages that have a genomic nucleic acid sequence as set forth in one of SEQ ID NOs: 5-9. The functional homologs are essentially capable of infecting bacteria of the same species (or strain) as that which is infected by the bacteriophage having a genomic nucleic acid sequence as set forth in one of SEQ ID NOs: 5-9. Typically, the functional homologs have a nucleic acid sequence that is at least 50% identical, 60% identical, 70% identical, 80% identical, 85% identical, 90% identical, at least 91% identical, at least 92% identical, at least 93% identical, at least 94% identical, at least 95% identical, 96% identical, 97% identical 98% identical, 99% identical, 99.5%, 99.9% identical to the genome having a nucleic acid sequence of SEQ ID NOs: 5 or 6; or at least 85% identical, 90% identical, 95% identical, 99% identical, 99.1% identical, 99.2% identical, 99.3% identical, 99.4% identical, 99.5% identical, 99.6% identical, 99.7% identical, 99.8% identical, or 99.9% identical to the genome having a nucleic acid sequence of SEQ ID NOs: 7, 8, or 9.

As used herein the terms “functional homolog”, “functionally homologous”, “variant”, or grammatical equivalents are used interchangeably herein and refer to a bacteriophage with a genomic nucleic acid sequence different than that of the sequenced (parent) bacteriophage (e.g., carrying at least one mutation) resulting in a bacteriophage that is endowed with substantially the same ensemble of biological activities (+10%, 20%, 40%, 50%, or 60% when tested under the same conditions) as that of the sequenced bacteriophage and can be classified as infecting essentially the same strain or species of bacteria based on known methods of species/strain classifications.

As would be appreciated by a person of skills in the art, bacteriophages “infect” bacteria by entering the bacterial cells and replicating in them.

In one embodiment of the invention, the viral nucleic acid integrates into the bacterial genome (i.e., the lysogenic pathway).

According to a particular embodiment of the invention, the bacteriophages disclosed herein lyse their target bacteria.

According to some embodiments, the genomic nucleic acid sequence of the bacteriophages described herein has a sequence that is at least 85% identical, 90% identical, at least 91% identical, at least 92% identical, at least 93% identical, at least 94% identical, at least 95% identical, 96% identical, 97% identical 98% identical, 99% identical, 99.1% identical, 99.2% identical, 99.3% identical, 99.4% identical, 99.5% identical, 99.6% identical, 99.7% identical, 99.8% identical or 99.9% identical to the sequence as set forth in SEQ ID NOs: 5, 6, 7, 8, or 9.

In particular, the bacteriophage has a genomic nucleic acid sequence at least 95% identical (or 95% homologous) to SEQ ID NOs: 5 or 6, or at least 99% identical (or 99% homologous) to SEQ ID NOs: 7, 8, or 9.

According to a specific embodiment of the invention, the bacteriophage has a genomic nucleic acid sequence at least 99% identical (or 99% homologous) to the full-length nucleic acid sequence as set forth in SEQ ID NOs: 5, 6, 7, 8 or 9.

As used herein, the terms “percent homology”, “percent identity”, “sequence identity”, “identity”, or grammatical equivalents thereof refer to a comparison between two nucleic acid or polypeptide sequences, particularly to an alignment between the residues of the two sequences. When percentage of sequence identity is used in reference to proteins it is recognized that residue positions that are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG.

Percent identity can be determined using any homology comparison software, including, for example, the BLASTN software of the National Center of Biotechnology Information (NCBI) such as by using default parameters.

It will be appreciated that the percent homology is not altered if an insertion is added (either a single insertion or a combination of insertions) to the phage genome. Thus, a bacteriophage comprising a genome that is at least 85% homologous to one of the sequences disclosed herein, which has been genetically modified to express a heterologous gene, is still contemplated by the present invention. This is true even if together with the heterologous gene, the percent homology of the modified genome is less than 85% homologous, as long as the aligned sequences remain at least 85% homologous.

Other exemplary sequence alignment programs that may be used to determine percent homology or identity between two sequences include, but are not limited to, the FASTA package (including rigorous (SSEARCH, LALIGN, GGSEARCH, and GLSEARCH) and heuristic (FASTA, FASTX/Y, TFASTX/Y, and FASTS/M/F) algorithms, the EMBOSS package (Needle, stretcher, water, and matcher), the BLAST programs (including, but not limited to BLASTN, BLASTX, TBLASTX, BLASTP, TBLASTN), megablast, DIAMOND, and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches, and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

According to some embodiments of the invention, the identity is a global identity, i.e., an identity over the entire nucleic acid sequences of the invention and not over portions thereof.

According to an additional or alternative embodiment, a functional homolog is determined as the average nucleotide identity (ANI), which is known to detect the DNA conservation of the core genome. In some embodiments, the ANI between the functional homolog and the bacteriophage having a genome as set forth in any one of SEQ ID NOs: 5, 6, 7, 8, or 9 is of at least about 95%, at least about, 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, a functional homolog is determined by the degree of relatedness between the functional homolog and the bacteriophage having a genome as set forth in any one of SEQ ID NO: 5, 6, 7, 8, or 9 determined as the Tetranucleotide Signature Frequency Correlation Coefficient, which is based on oligonucleotide frequencies. In some embodiments, the Tetranucleotide Signature Frequency Correlation coefficient between the variant (functional homolog) and the bacteriophage having a genome as set forth in any one of SEQ ID NO: 5, 6, 7, 8, or 9 is of about 0.99, 0.999 or more.

According to an additional or alternative embodiment, the degree of relatedness between the functional homolog (variant) and the bacteriophage having a genome as set forth in any one of SEQ ID NO: 5, 6, 7, 8, or 9 is determined as the degree of similarity obtained when analyzing the genomes of the parent and of the variant bacteriophage by Pulsed-Field Gel Electrophoresis (PFGE) using one or more restriction endonucleases. The degree of similarity obtained by PFGE can be measured by the Dice similarity coefficient.

In some embodiments, the Dice similarity coefficient between the variant and the bacteriophage having a genome as set forth in any one of SEQ ID NO: 5, 6, 7, 8, or 9 is of at least about 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, the degree of relatedness between the functional homolog and the bacteriophage having a genome as set forth in any one of SEQ ID NO: 5, 6, 7, 8, or 9 is determined by the Pearson correlation coefficient obtained by comparing the genetic profiles of both phages obtained by Repetitive Extragenic Palindromic element-based PCR (REP-PCR). In some embodiments, the Pearson correlation coefficient obtained by comparing the REP-PCR profiles of the variant and the parent bacteriophage is at least about 0.99, at least about 0.999, or more.

According to an additional or alternative embodiment, the degree of relatedness between the functional homolog and the bacteriophage having a genome as set forth in any one of SEQ ID NO: 5, 6, 7, 8, or 9 is defined by the linkage distance obtained by comparing the genetic profiles of both phages obtained by Multi-locus sequence typing (MLST). In some embodiments, the linkage distance obtained by MLST of the functional homolog and the bacteriophage having a genome as set forth in any one of 5, 6, 7, 8, or 9 is of at least about 0.99, at least about 0.999 or more.

According to an additional or alternative embodiment, the functional homolog comprises a functionally conserved gene or a fragment thereof, e.g., an integrase gene, a polymerase gene, a capsid protein assembly gene, a DNA terminase gene, a tail fiber gene, or a repressor gene that is at least about 97%, at least about 98%, at least about 99%, at least about 99.1%, at least about 99.5%, at least about 99.6%, at least about 99.7%, at least about 99.8%, at least about 99.9%, or more identical to that of the bacteriophage having a genome as set forth in any one of SEQ ID NO: 5, 6, 7, 8, or 9.

According to one embodiment of the invention, the functional homolog is defined by a comparison of the coding sequence (gene) order. According to another embodiment of the invention, the functional homolog is defined by a comparison of the order of non-coding sequences. According to yet another embodiment of the invention, the functional homolog is defined by a comparison of the order of coding and non-coding sequences.

As used herein, the term “combined coding region” refers to a nucleic acid sequence that includes all of the coding regions of the original bacteriophage, without the non-coding regions (e.g., regulatory elements, and the like) of the original bacteriophage.

The term “combined non-coding region” refers to a nucleic acid sequence that includes all of the non-coding regions of the original bacteriophage without the coding regions of the original bacteriophage.

According to some embodiments of the invention, the combined coding region of the functional homolog is such that it maintains the original order of the coding regions as within the genomic sequence of the bacteriophage having a genome as set forth in any one of SEQ ID NO: 5, 6, 7, 8, or 9.

For example, in case the genomic sequence has the following coding regions, A, B, C, D, E, F, and G, each flanked by non-coding sequences, the combined coding region will include a single nucleic acid sequence having the A+B+C+D+E+F+G coding regions combined together while maintaining the original order of their genome.

According to other embodiments of the invention, the combined non-coding region of the functional homolog is such that it maintains the original order of the non-coding regions as within the genomic sequence of the bacteriophage having a genome as set forth in any one of SEQ ID NO: 5, 6, 7, 8, or 9.

According to further embodiments of the invention, the combined non-coding region and coding region (i.e., the genome) of the functional homolog is such that it maintains the original order of the coding and non-coding regions as within the genomic sequence of the bacteriophage having a genome as set forth in any one of SEQ ID NO: 5, 6, 7, 8, or 9.

As used herein, the term “maintains” relates to at least about 90%, 91%, 92%, 93%, 94%, 95%, 96%, 97%, 98%, 99%, or 100% of the coding and/or non-coding regions of the functional homolog compared to the bacteriophage having a genome as set forth in any one of SEQ ID NO: 5, 6, 7, 8, or 9.

According to an additional or alternative embodiment, the functional homolog is defined by a comparison of gene content.

According to a specific embodiment, the functional homolog comprises a combined coding region that is at least about 90%, at least about 91%, at least about 92%, at least about 93%, at least about 94%, at least about 95%, at least about 96%, at least about 97%, at least about 98%, at least about 99%, or more (e.g., 100%) identical to the combined coding region existing in the genome of the bacteriophage having a genome as set forth in any one of SEQ ID NO: 5, 6, 7, 8, or 9.

In one embodiment of the invention, the bacteriophages show up to 85%, 86%, 87%, 88%, 89%, 90%, 91%, 92%, 93%, 94%, 95%, 96%, 97%, 98%, or 99% sequence identity with the bacteriophages disclosed herein and share similar host range and/or similar type of infectivity (i.e., lytic or temperate).

In another embodiment of the invention, the bacteriophages show up to 85%, 86%, 87%, 88%, 89%, 90%, 91%, 92%, 93%, 94%, 95%, 96%, 97%, 98%, or 99% sequence identity with the bacteriophages disclosed herein and have similar host range and similar type of infectivity as the bacteriophages disclosed herein.

Additional bioinformatics methods that may be used to determine relatedness between two bacteriophage genomes include Nucmer and Minimap (as examples of alignment-based tools); Jacard distance and MinHash (as examples of information-based tools); Codon usage similarity, pathway similarity, and protein motif similarity.

As used herein, the term “host range” refers to the bacteria that are susceptible to infection by a particular phage. The host range of a bacteriophage may include, but is not limited to, a strain, a subspecies, a species, a genus, or multiple genera of bacteria.

Bacteriophage isolates may be prepared and phenotyped using methods known in the art, e.g., a plaque assay, liquid media assay, and solid media assay. In some embodiments, the solid media assays to quantify and isolate bacteriophage are based on plaque assays, ranging from efficiency of plating (EOP) to spot testing. In some embodiments, the plate format used for the plaque assay can be modified, e.g., from a petri dish to a 48-well plate.

In some embodiments, infectivity is determined by the plaque presence in a solid assay only. In other embodiments, infectivity is determined by the plaque presence in a liquid assay only. In further embodiments, infectivity is determined by the plaque presence in both the liquid assay and the solid assay.

In one embodiment of the invention, the bacteriophages that target the plant pathogens of the genus Xanthomonas may do so by attaching to components of bacterial type 2 secretion system. Exemplary components of the bacterial type 2 secretion system are known in the art.

Thus, according to another aspect of the present invention, there is provided an agricultural composition comprising:
(i) at least one isolated bacteriophage capable of infecting the plant pathogen of the genus Xanthomonas, which attaches to components of bacterial type 2 secretion system; and
(ii) an agriculturally acceptable carrier,
wherein the composition comprises no more than 10 different strains of bacteriophage.

In one embodiment of the invention, the isolated bacteriophage has a genomic nucleic acid sequence at least 99% identical to the nucleic acid sequence as set forth in SEQ ID NOs: 5, 6, 7, 8, or 9.

The bacteriophages described herein are typically present in a preparation in which their prevalence (i.e., concentration) is enriched over (i.e., exceeds) that found in nature. According to one embodiment of the invention, the bacteriophages are present in the composition at a working concentration (i.e., effective amount) of >1×10⁸ plaque-forming units (PFU)/ml. According to a specific embodiment, the working concentration of the bacteriophages ranges between 5×10⁹ PFU/ml and 5×10¹⁰ PFU/ml. According to another specific embodiment, the working concentration of the bacteriophages in the composition is about 1×10¹⁰ PFU/ml.

In another aspect of the present invention, there is provided an agricultural composition comprising:
(i) at least one isolated bacteriophage capable of infecting the plant pathogen of the genus Xanthomonas, the at least one bacteriophage having a genomic nucleic acid sequence at least 85% identical to the nucleic acid sequence as set forth in SEQ ID NOs: 5, 6, 7, 8, or 9; and
(ii) an agriculturally acceptable carrier,
wherein the composition comprises no more than 10 different strains of bacteriophage.

Since bacteriophages infect bacterial cells, they may be found in specimens or samples that are rich in bacteria, e.g., environmental samples such as soil, water, and other materials. According to some embodiments of the invention, the agricultural composition (i.e. preparation) comprises less than 50 microbial species, e.g., bacteria and/or fungi—e.g., less than 40 bacterial species, less than 30 bacterial species, less than 20 bacterial species, less than 10 bacterial species, less than 5 bacterial species, less than 4 bacterial species, less than 3 bacterial species, less than 2 bacterial species or even completely devoid of bacteria.

According to a particular embodiment, the agricultural composition comprises a single strain of bacteriophage (or a functional homolog thereof), no more than two different bacteriophage strains (or functional homologs thereof), no more than three different bacteriophage strains (or functional homologs thereof), no more than four different bacteriophage strains (or functional homologs thereof), no more than five different bacteriophage strains (or functional homologs thereof), no more than six different bacteriophage strains (or functional homologs thereof), no more than seven different bacteriophage strains (or functional homologs thereof), no more than eight different bacteriophage strains (or functional homologs thereof), no more than nine different bacteriophage strains (or functional homologs thereof) or no more than ten different bacteriophage strains (or functional homologs thereof).

In a particular embodiment of the invention, the agricultural composition comprises no additional bacteriophage that infects plant pathogens other than Pseudomonas syringae pv. tomato or Xanthomonas species.

In another particular embodiment of the invention, the preparation comprises no additional bacteriophage that infects non-plant pathogens (e.g., bacteria that cause disease in humans).

In some embodiments, the present invention discloses agricultural compositions comprising bacteriophages that are directed only to species of the genus Xanthomonas.

Particular combinations of bacteriophages that are directed only to species of the genus Xanthomonas, according to one embodiment of the invention, include:
1. a phage having a genomic nucleic acid sequence at least 80% identical, 85% identical, or 90% identical to the nucleic acid sequence as set forth in SEQ ID NO: 5 and a phage having a genomic nucleic acid sequence at least 80% identical, 85% identical, or 90% identical to the nucleic acid sequence as set forth in SEQ ID NO: 6;
2. a phage having a genomic nucleic acid sequence at least 80% identical, 85% identical, or 90% identical to the nucleic acid sequence as set forth in SEQ ID NO: 5 and a phage having a genomic nucleic acid sequence at least 99% identical to the nucleic acid sequence as set forth in SEQ ID NO: 7;
3. a phage having a genomic nucleic acid sequence at least 80% identical, 85% identical, or 90% identical to the nucleic acid sequence as set forth in SEQ ID NO: 5 and a phage having a genomic nucleic acid sequence at least 99% identical to the nucleic acid sequence as set forth in SEQ ID NO: 8;
4. a phage having a genomic nucleic acid sequence at least 80% identical, 85% identical, or 90% identical to the nucleic acid sequence as set forth in SEQ ID NO: 5 and a phage having a genomic nucleic acid sequence at least 99% identical to the nucleic acid sequence as set forth in SEQ ID NO: 9;
5. a phage having a genomic nucleic acid sequence at least 80% identical, 85% identical, or 90% identical to the nucleic acid sequence as set forth in SEQ ID NO: 6 and a phage having a genomic nucleic acid sequence at least 99% identical to the nucleic acid sequence as set forth in SEQ ID NO: 7;
6. a phage having a genomic nucleic acid sequence at least 80% identical, 85% identical, or 90% identical to the nucleic acid sequence as set forth in SEQ ID NO: 6 and a phage having a genomic nucleic acid sequence at least 99% identical to the nucleic acid sequence as set forth in SEQ ID NO: 8;
7. a phage having a genomic nucleic acid sequence at least 80% identical, 85% identical, or 90% identical to the nucleic acid sequence as set forth in SEQ ID NO: 6 and a phage having a genomic nucleic acid sequence at least 99% identical to the nucleic acid sequence as set forth in SEQ ID NO: 9;
8. a phage having a genomic nucleic acid sequence at least 80% identical, 85% identical, or 90% identical to the nucleic acid sequence as set forth in SEQ ID NO: 5, a phage having a genomic nucleic acid sequence at least 80% identical, 85% identical, or 90% identical to the nucleic acid sequence as set forth in SEQ ID NO: 6, and a phage having a genomic nucleic acid sequence at least 99% identical to the nucleic acid sequence as set forth in SEQ ID NO: 7;
9. a phage having a genomic nucleic acid sequence at least 80% identical, 85% identical, or 90% identical to the nucleic acid sequence as set forth in SEQ ID NO: 5, a phage having a genomic nucleic acid sequence at least 80% identical, 85% identical, or 90% identical to the nucleic acid sequence as set forth in SEQ ID NO: 6, and a phage having a genomic nucleic acid sequence at least 99% identical to the nucleic acid sequence as set forth in SEQ ID NO: 8; and
10. a phage having a genomic nucleic acid sequence at least 80% identical, 85% identical, or 90% identical to the nucleic acid sequence as set forth in SEQ ID NO: 5, a phage having a genomic nucleic acid sequence at least 80% identical, 85% identical, or 90% identical to the nucleic acid sequence as set forth in SEQ ID NO: 6, and a phage having a genomic nucleic acid sequence at least 99% identical to the nucleic acid sequence as set forth in SEQ ID NO: 9.

Also contemplated are articles of manufacture and/or agricultural compositions, which comprise bacteriophages directed to both Pseudomonas syringae pv. tomato and specific of the genus Xanthomonas, for the control of both bacterial speck and bacterial spot. The bacteriophages may be formulated in a single composition or may be packaged separately and enclosed in a single article of manufacture.

The term “Pseudomonas syringae pv. tomato” relates to the gram-negative bacterial pathogen which is the causal agent of bacterial speck of tomato. According to some classifications, it belongs to the species P. syringae genospecies III. Exemplary strains of Pseudomonas syringae pv. tomato that are infected by the bacteriophage strains of the present invention are those that are found in environmental specimens, including, but not limited to, soil and water.

Thus, the articles of manufacture or agricultural compositions described herein may further comprise bacteriophages capable of infecting at least one, two, three, four, five, six, seven, eight, nine, or more Pseudomonas syringae pv. tomato strains that are causative for bacterial speck disease.

Examples of bacteriophage strains that infect Pseudomonas syringae pv. tomato include those described in PCT Application No. IL2022/050896, the contents of which are incorporated by reference, and those designated Ps1 to Ps24 in Table 2 in the section titled EXAMPLES, sub-section “MATERIALS AND METHODS”.

According to a specific embodiment of the invention, bacteriophage strains that infect Pseudomonas syringae pv. tomato comprise a genomic nucleic acid sequence that is 100% identical or at least 85%, 86%, 87%, 88%, 89%, 90%, 91%, 92%, 93%, 94%, 95%, 96%, 97%, 98%, or 99% identical to the nucleic acid sequence as set forth in SEQ ID NO: 1, 2, 3, or 4.

An exemplary contemplated combination of phages includes a phage having a genomic nucleic acid sequence at least 80% identical, at least 85% identical, at least 90% identical, or 100% identical to the nucleic acid sequence as set forth in SEQ ID NO: 5, a phage having a genomic nucleic acid sequence at least 80% identical, at least 85% identical, at least 90% identical, or 100% identical to the nucleic acid sequence as set forth in SEQ ID NO: 6, a phage having a genomic nucleic acid sequence at least 85% identical or 100% identical to the nucleic acid sequence as set forth in SEQ ID NO: 1, and a phage having a genomic nucleic acid sequence at least 90% identical or 100% identical to the nucleic acid sequence as set forth in SEQ ID NO: 2.

Other exemplary combinations are provided in Table 1, herein below. It should be noted that combinations of bacteriophages having at least 85% identity to each of the SEQ ID NOs mentioned in Table 1 are also encompassed in the present invention.

TABLE 1
Exemplary combinations of phages.

| | Pseudomonas | Xanthomonas |
|---|---|---|
| | phages SEQ ID | phages SEQ |
| Combination | NO (% homology) | ID NO |
| A | 1, 2, 3 | 5, 6, 7 |
| B | 1, 2, 3 | 5, 6, 8 |
| C | 1, 2, 3 | 5, 6, 9 |
| D | 1, 2, 4 | 5, 6, 7 |
| E | 1, 2, 4 | 5, 6, 8 |
| F | 1, 2, 4 | 5, 6, 9 |
| G | 1, 2, 3 | 5, 7, 8 |
| H | 1, 2, 3 | 5, 7, 9 |
| I | 1, 2, 4 | 5, 7, 8 |
| J | 1, 2, 4 | 5, 7, 9 |
| K | 1, 2, 3, 4 | 5, 7, 8, 9 |

The bacteriophage compositions can be applied to plants by spraying, fogging, dusting, coating, soaking, irrigating, drenching, or other ways of administering them with the active ingredients. Alternatively, the bacteriophage compositions can be applied to the plant's seeds, the soil around the plant, or the soil, rice pads, or the water for hydroponic culture where the seeds are to be sown. The treatment may be applied either before (preventatively) or after the plant is infected with the pathogen.

According to one embodiment of the invention, the treatment regimen is performed such as to control the spread of the pathogen and/or eliminate the pathogen and/or eliminate/reduce/minimize any damage that can be caused by the pathogen.

According to some embodiments, the bacteriophages can be formulated as dips, sprays, seed coatings, or concentrates.

In a specific embodiment of the invention, the bacteriophages are provided as a dried formulation—e.g., powder. In another specific embodiment of the invention, the bacteriophages are provided in a liquid solution.

It will be appreciated that by controlling (i.e., reducing) pathogenic infection in the plant, the post-harvest quality of the plant is improved and losses are reduced. Concurrently or alternatively, plant performance parameters may also be improved (including, but not limited to growth parameters, crop production, and pathogenic resistance).

The contacting of the plant with the bacteriophage may be effected at any stage of the plant's life cycle e.g. a post-blossom stage, a blossom stage, a pre-blossom stage, or any combination thereof.

In one embodiment, the contacting may be carried out at the pre-germination phase. In another embodiment, the contacting is carried out at the post-germination phase. In a specific embodiment, the contacting is effected at or after the seedling stage (e.g. 4 or more leaves stage).

The contacting may be effected in the vicinity of or onto a plant part, such as a shoot, root, stalk, stem, trunk, seed, fruit, flower, leaf, or any combination thereof of the plant.

According to an embodiment, the bacteriophage treatment is applied in an open field. According to another embodiment, the bacteriophage treatment is applied in a greenhouse or a nursery.

According to an embodiment, the bacteriophage treatment is applied once. According to another embodiment, the bacteriophage treatment is applied at least twice at any regimen or duration.

According to some embodiments, the bacteriophage treatment comprises repeated applications (2 or more applications e.g., every day, every week, seasonal, bi-weekly, bi-monthly, etc.). Repeated applications are especially envisaged for field and greenhouse treatments.

According to one embodiment of the invention, repeated applications comprise daily, weekly, twice a week, every other week, or monthly administration during blossom, post-blossom, pre-blossom, or any combination thereof. An exemplary regimen may include, but is not limited to, spraying the composition comprising at least one bacteriophage of the present invention on plants in open fields and greenhouses, or adding said composition to the irrigation system or means used for irrigating plants grown in open fields, greenhouses, nurseries, and pots. In a specific embodiment of the invention, the composition is applied twice a week.

In one embodiment of the invention, the contacting is not carried out during daylight hours. This is because applying the phages to plants in the evening results in longer phage persistence in the phyllosphere, thereby giving the phages a prolonged time window to infect and kill their bacterial targets.

In an additional or alternative embodiment of the invention, the contacting is effected in the absence of a copper-based product.

The bacteriophages disclosed herein may be provided with or without an agriculturally acceptable carrier.

As used herein the term “agriculturally acceptable carrier” refers to a material that facilitates the application of an active bacteriophage to the intended target, which may be for example a plant, a plant material, compost, earth, surroundings or equipment, or that facilitates storage, transport or handling. Carriers used in compositions for application to plants and plant material are preferably non-phytotoxic or only mildly phytotoxic. Preferably, the carrier does not reduce the infectivity of the bacteriophage. A suitable carrier may be a solid, liquid, or gas depending on the desired formulation. In one embodiment of the invention, the carriers include polar liquid carriers such as water, mineral oils, and vegetable oils. In one embodiment the carrier enhances the stability of the active ingredient as described herein.

In one embodiment of the invention, the carrier includes a dispersant, a surfactant, an additive, water, a thickener, an anti-caking agent, residue breakdown, a composting formulation, a granular application, diatomaceous earth, an oil, a coloring agent, a stabilizer, a tackifier, a preservative, a polymer, a coating, an adjuvant, a salt, an anti-complexing agent, or any combination thereof. One of ordinary skills in the art can readily determine the appropriate carrier to be used taking into consideration factors such as a particular bacteriophage strain, the specific plant to which the bacteriophage is to be applied, the type of soil, climate conditions, whether the bacteriophage is in liquid, solid or powder form, and the like.

In another embodiment of the invention, the additive comprises an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or any combination thereof.

In a further embodiment of the invention, the surfactant contains heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or any combination thereof.

In yet another embodiment of the invention, the anti-caking agent includes a sodium salt, such as a sodium sulfite, a sodium sulfate, a sodium salt of monomethyl naphthalene sulfonate, a sodium salt of dimethyl naphthalene sulfonate, or any combination thereof, or a calcium salt, such as calcium carbonate, diatomaceous earth, or any combination thereof.

In some embodiments, the composition is devoid of a copper-based element.

Exemplary agriculturally acceptable carriers include, but are not limited to, vermiculite, charcoal, sugar factory carbonation press mud, rice husk, carboxymethyl cellulose, peat, perlite, fine sand, calcium carbonate, flour, alum, a starch, talc, polyvinyl pyrrolidone, or a combination thereof.

In some embodiments, the bacteriophages are prepared as solid, liquid, emulsion, or powdered formulations as is known in the art. The compositions of the present invention can be formulated as a seed coating formulation, a liquid formulation for application to plants or to a plant growth medium, or a solid formulation for application to plants or to a plant growth medium.

When the bacteriophage is prepared as a liquid formulation for application to plants or to a plant growth medium, it can be prepared in a concentrated formulation or a working form formulation. In one embodiment of the invention, formulations of the present invention are aqueous or oil-based solutions for application to seeds or foliage.

When the bacteriophage of the present invention is prepared as a solid formulation for application to plants or to a plant growth medium, it can be prepared as a granular formulation or a powder agent. The seed coating formulation can be a powder or granular formulation for application to seeds.

The bacteriophage composition can further include an agrochemical (i.e. an agent that promotes the growth of a plant and/or maintains the health of a plant). Examples of such agents include a fertilizer, a micronutrient fertilizer material, an insecticide, a herbicide, a plant growth regulator, an acaricide, a rodenticide, a fungicide, a nematicide, a nutrient, a molluscicide, an algicide, a pesticide, a fungal inoculant, or a combination thereof.

According to one embodiment, the fertilizer is a liquid fertilizer. Liquid fertilizer can include, without limitation, ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, calcium nitrate, dolomitic limestone, hydrated lime, calcium carbonate, diammonium phosphate, monoammonium phosphate, magnesium nitrate, magnesium sulfate, potassium nitrate, potassium chloride, potassium magnesium sulfate, potassium sulfate, sodium nitrates, magnesian limestone, magnesia, urea, urea-formaldehydes, urea ammonium nitrate, sulfur-coated urea, polymer-coated urea, isobutylidene diurea, K₂SO₄-2MgSO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion, or any combination thereof.

According to another embodiment of the invention, the micronutrient fertilizer material comprises boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate, or any combination thereof.

According to a further embodiment of the invention, the insecticide includes an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or any combination thereof.

According to yet a further embodiment of the invention, the herbicide comprises a chlorophenoxy compound, a nitrophenolic compound, a nitrocresolic compound, a dipyridyl compound, an acetamide, an aliphatic acid, an anilide, a benzamide, a benzoic acid, a benzoic acid derivative, anisic acid, an anisic acid derivative, a benzonitrile, benzothiadiazinone dioxide, a thiocarbamate, a carbamate, a carbanilate, chloropyridinyl, a cyclohexenone derivative, a dinitroaminobenzene derivative, a fluorodinitrotoluidine compound, isoxazolidinone, nicotinic acid, isopropylamine, an isopropylamine derivative, oxadiazolinone, a phosphate, a phthalate, a picolinic acid compound, a triazine, a triazole, a uracil, a urea derivative, endothall, sodium chlorate, or any combination thereof.

According to another embodiment of the invention, the fungicide comprises a substituted benzene, a thiocarbamate, an ethylene bis dithiocarbamate, a thiophthalidamide, a copper compound, an organomercury compound, an organotin compound, a cadmium compound, anilazine, benomyl, cycloheximide, dodine, etridiazole, iprodione, metalaxyl, thiamimefon, triforine, or a combination thereof.

According to yet another embodiment of the invention, the fungal inoculant comprises a fungal inoculant of the family Glomeraceae, a fungal inoculant of the family Claroidoglomeraceae, a fungal inoculant of the family Gigasporaceae, a fungal inoculant of the family Acaulosporaceae, a fungal inoculant of the family Sacculosporaceae, a fungal inoculant of the family Entrophosporaceae, a fungal inoculant of the family Pacidsporaceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporaceae, a fungal inoculant of the family Scutellosporaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, or any combination thereof.

In one embodiment of the embodiment, the plant growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl, and uniconazole. Other examples of plant growth regulators that can be comprised in the article of manufacture include those based on dichlorophene and benzylalcohol hemi formal (Proxel® from ICI or Acticide® RS from Thor Chemie and Kathon® MK from Rohm & Haas) and isothiazolinone derivatives, such as alkylisothiazolinones and benzisothiazolinones (Acticide® MBS from Thor Chemie). Other plant growth regulators that can be incorporated seed coating compositions are described in US 2012/0108431, which is incorporated by reference in its entirety.

Examples of nematode-antagonistic biocontrol agents include, but are not limited to, ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. In a particular embodiment of the invention, the nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergillus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens, and Rhizobacteria.

In another embodiment of the invention, the bacteriophage preparation further comprises a nutrient. In a specific embodiment, the nutrient is selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, and Potassium nitrate. Such compositions can exist as free salts or ions within the seed coat composition. Alternatively, nutrients/fertilizers can be complexed or chelated to provide sustained release over time.

In yet another embodiment of the invention, the preparation may comprise a rodenticide selected from the group of substances consisting of 2-isovalerylindan-1,3-dione, 4-(quinoxalin-2-yl-amino)benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide

As used herein, the term “about” refers to +10% of the indicated value.

The terms “comprises”, “comprising”, “includes”, “including”, “having”, and their conjugates mean “including but not limited to”.

The term “consisting of” means “including and limited to”.

The term “consisting essentially of” means that the composition, method, or structure may include additional ingredients, steps, and/or parts, but only if the additional ingredients, steps, and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

As used herein, the term “method” refers to manners, means, techniques, and procedures for accomplishing a given task including, but not limited to, those manners, means, techniques, and procedures either known to or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological, and recombinant DNA techniques. Such techniques are thoroughly explained in the literature.

### Materials and Methods

Environmental samples were taken from fields in which tomatoes were grown intensively (CF), organic farming where tomatoes grow without copper-based bactericides (OF), effluents of a municipal waste plant (MW), and a municipal wastewater treatment plant where raw sewage was sampled (RS). CF and OF soil samples were suspended in up to 50 ml Nutrient Broth medium (NB) (Formedium, Norfolk, UK) and liquid samples (MW and RS) were mixed in equal volumes (1:1) of NB and samples. The mixture was incubated overnight and was then centrifuged twice at 3000 RCF for 10 minutes following filtration through 0.45 μm and 0.22 μm syringe filters. The liquid, named lysate, was then serially diluted and each dilution was incubated with 200 μl target bacteria (after being cultured overnight) at an optical density at 600 nm (OD₆₀₀) of 1.5 and plated on top agar (namely, drop assay).

The resulting plates were incubated overnight and on the following day, the bacteria were screened for single plaques.

Host range: Bacterial isolates of P. syringae and Xanthomonas were collected from numerous bacterial collections (Table 2) that were used as control. It should be noted that the bacterial collections may also include bacteria that are considered beneficial for plants. Each of the isolated phages was subjected to a host range test using a BioTek 800 TS absorbance reader (BioTek, Winooski, VT, USA). Each bacterium was grown in NB with the given bacteriophage and the growth curve was measured to detect the bacteria from the collection that is affected by the bacteriophage. In addition, each phage was tested against all bacteria by drop assay (from the isolated species, either Pst or all Xanthomonas species) to evaluate the host range on solid media.

TABLE 2
Bacterial collection used to isolate and
test environmentally collected bacteriophages.

| Bacterial | | Vendor’s catalog | |
|---|---|---|---|
| name | Commercial source | name/number | Strain |
| Xa1 | NCPPB | 195 | Xanthomonas |
| | | | vesicatoria (XV) |
| Xa2 | NCPPB | 509 | XV |
| Xa3 | NCPPB | 975 | XV |
| Xa4 | NCPPB | 2576 | XV |
| Xa5 | NCPPB | 2595 | XV |
| Xa6 | NCPPB | 3240 | XV |
| Xa7 | NCPPB | 3311 | XV |
| Xa8 | NCPPB | 3314 | XV |
| Xa9 | NCPPB | 4321 | XP |
| Xa10 | NCPPB | 4322 | XP |
| Xa11 | NCPPB | 4323 | Xanthomonas |
| | | | gardneri (XG) |
| Xa13 | NCPPB | 1421 | XV |
| Xa14 | NCPPB | 2574 | XV |
| Xa15 | NCPPB | 3800 | XV |
| Xa16 | NCPPB | 3614 | XV |
| Xa17 | NCPPB | 4324 | XG |
| Xa18 | NCPPB | 2573 | Xanthomonas |
| | | | euvesicatoria |
| | | | pv. |
| | | | euvesicatoria |
| | | | (XE) |
| Xa19 | NCPPB | 941 | XV |
| Xa20 | NCPPB | 3801 | XV |
| Xa21 | ATCC | 981 | XG |
| Xa22 | ATCC | 11633 | XE |
| Xa23 | ATCC | 35937 | XV |
| Xa24 | NSW Plant | DAR 80973 | XV |
| | Pathology & | | |
| | Mycology | | |
| | Herbarium | | |
| Ps1 | NCPPB | 269 | Pseudomonas |
| | | | syringae pv. |
| | | | tomato (Pst) |
| Ps2 | NCPPB | 878 | Pst |
| Ps3 | NCPPB | 996 | Pst |
| Ps4 | NCPPB | 1008 | Pst |
| Ps5 | NCPPB | 1106 | Pst |
| Ps6 | NCPPB | 1368 | Pst |
| Ps7 | NCPPB | 2424 | Pst |
| Ps8 | NCPPB | 2683 | Pst |
| Ps9 | NCPPB | 3333 | Pst |
| Ps10 | NCPPB | 3645 | Pst |
| Ps11 | NCPPB | 3784 | Pst |
| Ps12 | NCPPB | 4369 | Pst |
| Ps13 | ATCC | DC3000 | Pst |
| Ps14 | ATCC | 10862 | Pst |
| Ps16 | NRRL (The Agriculture | B-883 | Pst |
| | Research Service Culture | | |
| | Collection), USA | | |
| Ps18 | NARO (National | 302665 | Pst |
| | Agriculture and Food | | |
| | Research Organization), | | |
| | Japan | | |
| Ps19 | NARO (National | 301591 | Pst |
| | Agriculture and Food | | |
| | Research Organization), | | |
| | Japan | | |
| Ps20 | The ICMP (International | ICMP 9324 | Pst |
| | Collection of | | |
| | Microorganisms from | | |
| | Plants), NZ | | |
| Ps21 | The ICMP (International | ICMP 4933 | Pst |
| | Collection of | | |
| | Microorganisms from | | |
| | Plants), NZ | | |
| Ps22 | The ICMP (International | ICMP 9499 | Pst |
| | Collection of | | |
| | Microorganisms from | | |
| | Plants), NZ | | |
| Ps23 | The ICMP (International | ICMP 2843 | Pst |
| | Collection of | | |
| | Microorganisms from | | |
| | Plants), NZ | | |
| Ps24 | NSW Plant | DAR 75965 | Pst |
| | Pathology & | | |
| | Mycology | | |
| | Herbarium | | |
| npPs1 | ATCC | 27853 | Pseudomonas |
| | | | aeruginosa |
| npPs2 | DSMZ | 14936 | Pseudomonas |
| | | | pode |
| npPs3 | DSMZ | 50083 | Pseudomonas |
| | | | chlororaphis |
| npPs4 | DSMZ | 19095 | Pseudomonas |
| | | | protegens |
| npPs5 | DSMZ | 6698 | Pseudomonas |
| | | | chlororaphis |
| | | | subsp. |
| | | | Aureofaciens |

np = non-pathogenic

Assessment of bacterial spot and speck symptoms inhibition in tomato plants: An in-planta experiment was carried out in a commercial plot c.v. 8575 in Dolores Hidalgo, Guanajuato in the state of Guanajuato at the coordinates: 21° 07′ 05.6″ N-100° 55′ 00.7″ W, Mexico, in which tomato plants affected by Xanthomonas campestris p.v. vesicatoria (Xcv) and Pseudomonas syringae p.v. tomato (Pst) were grown. During the August-September season of 2023, the plants were treated with either a bacteriophage cocktail (10.3-11.02 L/ha) or Cupravit® Hydro (2.5 kg/ha, prepared according to the manufacturer's instructions). Each treatment consisted of 12 applications (2 per week) with an average water consumption of 551 L of water per hectare.

Disease severity index in each plant was determined once a week after the first treatment application on four leaflets on each plant based on the following scale: 0-without symptoms; 1-1% of the leaf area affected; 2-5% of the leaf area affected; 3-15% of the leaf area affected; 4-25% of the leaf area affected; and 5-50% of the leaf area affected.

Treatment efficacy was calculated according to Equation (1):

$$\frac{Control index - Treatment index}{Control index} \times 100 , \qquad Equation ( 1 )$$

wherein “control index” is the disease severity index determined for untreated control plants and “treatment index” is the disease severity index determined for treated plants.

Chlorophyll content was determined using an Amtast MC-100 chlorophyll meter in 25 leaflets of 5 plants randomly selected from each useful plot in each experimental unit. The chlorophyll content was measured at the apex of the foliage and the middle part of the leaflet. The results were expressed in SPAD (Soil Plant Analysis Development) units, which represent the difference between the transmittance of a red (650 nm) and an infrared (940 nm) light through the leaf.

### Example 1

Isolation of 5 Bacteriophages Active Against the Genus Xanthomonas and 4 Bacteriophages Active Against Pseudomonas syringae pv. Tomato

Most of the isolated bacteriophages were isolated several times from different environmental samples, in some cases from distinct geographical locations. However, several bacteriophages were distinct to the environments from which they were isolated (data not shown). Several of these phages had high genomic similarities and were therefore clustered into clades and only one representative was chosen. The representative phages were sequenced and named numerically and the best 9 isolates active against Pseudomonas or Xanthomonas species were selected for further study (Table 3). Eight of these phage isolates were deposited by the inventors with an International Depositary Authority (IDA) under the provisions of the Budapest Treaty on the International Recognition of the Deposit of Microorganisms for the Purpose of Patent Procedure and Regulations thereunder.

TABLE 3
Details of the representative 9 phage isolates.

| | | | | | IDA |
|---|---|---|---|---|---|
| Phage | SEQ | Bacterial host | Genome | | Accession no. |
| name | ID | (available as | size | Classification | (name of IDA; |
| (clade) | NO | deposit no.) | (bp) | (orders, families, races, or strains) | date of deposit) |
| Ep007 | 1 | Pst | 39435 | Viruses; Duplodnaviria; | F/00196 |
| (F) | | (ATCC BAA- | | Heunggongvirae; Uroviricota; | (PCM; Jul. 29, |
| | | 871TM) | | Caudoviricetes; Caudovirales; | 2022) |
| | | | | Autographiviridae; | |
| | | | | Studiervirinae; Ghunavirus; | |
| | | | | unclassified Ghunavirus | |
| Ep017 | 2 | Pst | 40570 | Viruses; Duplodnaviria; | F/00197 |
| (KB1) | | (ATCC BAA- | | Heunggongvirae; Uroviricota; | (PCM; Jul. 29, |
| | | 871TM) | | Caudoviricetes; Caudovirales; | 2022) |
| | | | | Autographiviridae; | |
| | | | | Studiervirinae; Ghunavirus; | |
| | | | | unclassified Ghunavirus | |
| Ep020 | 3 | Pst (ATCC | 98129 | Viruses; Duplodnaviria; | F/00198 |
| (AY2) | | BAA-871TM) | | Heunggongvirae; Uroviricota; | (PCM; Jul. 29, |
| | | | | Caudoviricetes; Caudovirales; | 2022) |
| | | | | Myoviridae; Otagovirus; | |
| | | | | unclassified Otagovirus | |
| Ep022 | 4 | Pst (B/00456) | 40665 | Viruses; Duplodnaviria; | F/00200 |
| (SH2) | | | | Heunggongvirae; Uroviricota; | (PCM; Jul. 29, |
| | | | | Caudoviricetes; Caudovirales; | 2022) |
| | | | | Autographiviridae; Studiervirinae; | |
| | | | | Ghunavirus; unclassified | |
| | | | | Ghunavirus | |
| Ep026 | 5 | Xan | 227329 | Viruses; Duplodnaviria; | F/00201 |
| (Q) | | | | Heunggongvirae; Uroviricota; | (PCM; Jun. 02, |
| | | | | Caudoviricetes; Unclassified | 2023 |
| | | | | Xanthomonas bacteriophage | |
| Ep025 | 6 | Xan | 197087 | Viruses; Duplodnaviria; | n/a |
| (N) | | | | Heunggongvirae; Uroviricota; | |
| | | | | Caudoviricetes; Caudovirales; | |
| | | | | Myoviridae; Menderavirus; | |
| | | | | unclassified Menderavirus | |
| Ep027 | 7 | Xan | 44425 | Viruses; Duplodnaviria; | F/00202 |
| (SH23) | | | | Heunggongvirae; Uroviricota; | (PCM; Jun. 02, |
| | | | | Caudoviricetes; Caudovirales; | 2023 |
| | | | | Autographiviridae; Pradovirus; | |
| | | | | unclassified Pradovirus | |
| Ep028 | 8 | Xan | 42944 | Viruses; Duplodnaviria; | F/00203 |
| (SH47) | | | | Heunggongvirae; Uroviricota; | (PCM; Jun. 02, |
| | | | | Caudoviricetes; Septimatrevirus ; | 2023 |
| | | | | unclassified Septimatrevirus | |
| Ep029 | 9 | Xan | 64119 | Viruses; Duplodnaviria; | I-6020 |
| (SH48) | | | | Heunggongvirae; Uroviricota; | (CNCM, |
| | | | | Caudoviricetes; | Dec. 05, |
| | | | | Mesyanzhinovviridae; | 2023) |
| | | | | Bradleyvirinae; Bosavirus ; | |
| | | | | unclassified Bosavirus | |

PCM = Polish Collection of Microorganisms, address: Institute of Immunology and Experimental Therapy,
Polish Academy of Sciences, ul. Weigla 12, 53-114 Wroclaw, Poland;
CNCM = National Collection of Cultures of Microorganisms, address: Institut Pasteur, 25-28 rue du
Docteur Roux 75724 Paris Cedex 15, France.

Single plaques were isolated by touching the plaque with a sterile tip, resuspending in 1 ml NB, and repeating the plaque overlay assay twice more as described above. At this point, plaques were considered to present a single bacteriophage and were propagated in 3 ml of NB in the presence of the target bacterium. Lysates were filter-sterilized and the bacteriophage titer was determined using a series of dilutions. DNA was extracted from the phages using the DNeasy Blood & Tissue Kit, Cat. No. 69504 (Qiagen, Germany), performing a DNAse I treatment step prior to the kit's protocol. The DNA was then sequenced using the Illumina Tagment DNA Enzyme and Buffer Kit (Illumina, USA).

Each of the sequences was analyzed using available homology comparison software to evaluate the coverage (the fraction of the hit sequence that aligns with the query sequence) and percent identity (the fraction of the alignment that is identical between the hit sequence and the query sequence) compared to a known phage sequence (“hit”). For each of the phages, the highest coverage and percent identity to a known sequence is presented in Table 4.

TABLE 4
Phage characterization.

| SEQ | Description (as described | | Query | % | Hit accession |
|---|---|---|---|---|---|
| ID | in the NCBI repository) | Scientific name | cover | identity | No. |
| 1 | Pseudomonas phage | Pseudomonas | 58% | 78.5% | NC_048200.1 |
| | sh12 genome assembly, | phage shl2 | | | |
| | chromosome: 1 | | | | |
| 2 | Pseudomonas | Pseudomonas | 94% | 98.02% | OK523998.1 |
| | phage Pst_GM1, | phage Pst_GM1 | | | |
| | complete genome | | | | |
| 3 | Pseudomonas | Pseudomonas | 30% | 83.27% | NC_023601.2 |
| | phage phiPsa374, | phage | | | |
| | complete genome | phiPsa374 | | | |
| 4 | Pseudomonas | Pseudomonas | 96% | 98.39% | KJ507099.1 |
| | phage phiPSA2, | phage phiPSA2 | | | |
| | complete genome | | | | |
| 5 | Select seq | — | 0% | 87.27% | CACVDZ010 |
| | CACVDZ010000001.1 | | | | 000001.1 |
| | uncultured phage | | | | |
| | genome assembly, contig: | | | | |
| | PH2015_14_scaffold_1_ | | | | |
| | prodigal-single, whole | | | | |
| | genome shotgun sequence | | | | |
| 6 | Xanthomonas phage BUDD, | Xanthomonas | 22% | 78.49% | ON758385.1 |
| | complete genome | phage BUDD | | | |
| 7 | Xanthomonas phage Pagan, | Xanthomonas | 97% | 94.25% | MK903278.1 |
| | complete genome | phage Pagan | | | |
| 8 | Xanthomonas phage vB_ | Xanthomonas | 96% | 97.52% | ON932082.1 |
| | Xar IVIA-DoCa8, | phage vB | | | |
| | complete genome | | | | |
| 9 | Xanthomonas phage | Select seq | 99% | 96.21% | OK275492.1 |
| | FMYAK-P1, | OK275492.1 | | | |
| | complete genome | Xanthomonas | | | |
| | | phage | | | |
| | | FMYAK-P1 | | | |

### Example 2

### Determining the Host Range of the 9 Phage Isolates

Bacteriophages from each phylogroup were tested against all bacteria using phage-bacteria interaction assay in both liquid broth and solid media. Pseudomonas and Xanthomonas phages covering the entire bacterial range (Table 5) were isolated. Several phages had an extremely broad host range, these included phage Ep007 (SEQ ID NO: 1) and Ep026 (SEQ ID NO: 5).

TABLE 5
Phage-Bacteria interaction.

| | Phage | | | | | | | | | |
|---|---|---|---|---|---|---|---|---|---|---|
| | SEQ | SEQ | SEQ | SEQ | SEQ | SEQ | SEQ | SEQ | SEQ | |
| Bacterium | 1 | 2 | 3 | 4 | 5 | 6 | 7 | 8 | 9 | Total |
| Xa1 | 0 | 0 | 0 | 0 | 1 | 0 | 0 | 0 | 0 | 1 |
| Xa2 | 0 | 0 | 0 | 0 | 1 | 0 | 0 | 0 | 0 | 1 |
| Xa3 | 0 | 0 | 0 | 0 | 1 | 1 | 0 | 1 | 1 | 1 |
| Xa4 | 0 | 0 | 0 | 0 | 1 | 0 | 0 | 1 | 1 | 1 |
| Xa5 | 0 | 0 | 0 | 0 | 0 | 1 | 0 | 0 | 0 | 1 |
| Xa6 | 0 | 0 | 0 | 0 | 1 | 1 | 1 | 0 | 1 | 1 |
| Xa7 | 0 | 0 | 0 | 0 | 1 | 1 | 0 | 0 | 0 | 1 |
| Xa8 | 0 | 0 | 0 | 0 | 1 | 0 | 0 | 1 | 1 | 1 |
| Xa9 | 0 | 0 | 0 | 0 | 1 | 1 | 0 | 0 | 0 | 1 |
| Xa10 | 0 | 0 | 0 | 0 | 1 | 0 | 0 | 0 | 0 | 1 |
| Xa11 | 0 | 0 | 0 | 0 | 1 | 1 | 0 | 0 | 1 | 1 |
| Xa13 | 0 | 0 | 0 | 0 | 0 | 1 | 1 | 0 | 1 | 1 |
| Xa14 | 0 | 0 | 0 | 0 | 1 | 0 | 1 | 1 | 1 | 1 |
| Xa15 | 0 | 0 | 0 | 0 | 1 | 1 | 0 | 0 | 0 | 1 |
| Xa16 | 0 | 0 | 0 | 0 | 1 | 0 | 1 | 1 | 1 | 1 |
| Xa17 | 0 | 0 | 0 | 0 | 1 | 0 | 0 | 0 | 1 | 1 |
| Xa18 | 0 | 0 | 0 | 0 | 1 | 0 | 0 | 0 | 0 | 1 |
| Xa19 | 0 | 0 | 0 | 0 | 1 | 0 | 0 | 1 | 1 | 1 |
| Xa20 | 0 | 0 | 0 | 0 | 1 | 1 | 0 | 1 | 1 | 1 |
| Xa21 | 0 | 0 | 0 | 0 | 1 | 1 | 0 | 0 | 1 | 1 |
| Xa22 | 0 | 0 | 0 | 0 | 1 | 1 | 0 | 0 | 1 | 1 |
| Xa23 | 0 | 0 | 0 | 0 | 1 | 0 | 0 | 0 | 0 | 1 |
| Xa24 | 0 | 0 | 0 | 0 | 1 | 0 | 0 | 1 | 0 | 1 |
| Ps1 | 1 | 1 | 1 | 1 | 0 | 0 | 0 | 0 | 0 | 1 |
| Ps2 | 0 | 1 | 0 | 0 | 0 | 0 | 0 | 0 | 1 | 1 |
| Ps3 | 1 | 1 | 1 | 1 | 0 | 0 | 0 | 0 | 0 | 1 |
| Ps4 | 1 | 0 | 1 | 1 | 0 | 0 | 0 | 0 | 0 | 1 |
| Ps5 | 1 | 1 | 1 | 1 | 0 | 0 | 0 | 0 | 1 | 1 |
| Ps6 | 1 | 1 | 0 | 1 | 0 | 0 | 0 | 0 | 0 | 1 |
| Ps7 | 1 | 1 | 0 | 1 | 0 | 0 | 0 | 0 | 0 | 1 |
| Ps8 | 1 | 0 | 1 | 1 | 0 | 0 | 0 | 0 | 1 | 1 |
| Ps9 | 1 | 1 | 1 | 1 | 0 | 0 | 0 | 0 | 1 | 1 |
| Ps10 | 1 | 1 | 0 | 0 | 0 | 0 | 0 | 0 | 0 | 1 |
| Ps11 | 0 | 1 | 1 | 1 | 0 | 0 | 0 | 0 | 1 | 1 |
| Ps12 | 1 | 1 | 1 | 1 | 0 | 0 | 0 | 0 | 0 | 1 |
| Ps13 | 1 | 1 | 0 | 1 | 0 | 0 | 0 | 0 | 0 | 1 |
| Ps14 | 1 | 1 | 1 | 1 | 0 | 0 | 0 | 0 | 0 | 1 |
| Ps15 | 1 | 1 | 0 | 0 | 0 | 0 | 0 | 0 | 0 | 1 |
| Ps16 | 1 | 1 | 1 | 1 | 0 | 0 | 0 | 0 | 0 | 1 |
| Ps18 | 1 | 1 | 1 | 1 | 0 | 0 | 0 | 0 | 0 | 1 |
| Ps19 | 1 | 1 | 1 | 1 | 0 | 0 | 0 | 0 | 0 | 1 |
| Ps20 | 1 | 1 | 1 | 1 | 0 | 0 | 0 | 0 | 0 | 1 |
| Ps21 | 0 | 1 | 1 | 1 | 0 | 0 | 0 | 0 | 0 | 1 |
| Ps22 | 1 | 1 | 1 | 1 | 0 | 0 | 0 | 0 | 0 | 1 |
| Ps23 | 1 | 1 | 1 | 1 | 0 | 0 | 0 | 0 | 0 | 1 |
| Ps24 | 1 | 1 | 1 | 1 | 0 | 0 | 0 | 0 | 0 | 1 |
| npPs1 | 0 | 0 | 0 | 0 | 0 | 0 | 0 | 0 | 0 | 0 |
| npPs2 | 0 | 0 | 0 | 0 | 0 | 0 | 0 | 0 | 0 | 0 |
| npPs3 | 0 | 0 | 0 | 0 | 0 | 0 | 0 | 0 | 0 | 0 |
| npPs4 | 0 | 0 | 0 | 0 | 0 | 0 | 0 | 0 | 0 | 0 |
| npPs5 | 0 | 0 | 0 | 0 | 0 | 0 | 0 | 0 | 0 | 0 |

1 = lysis of the bacterial strain by the isolated phage;
0 = no interaction

As shown in Table 5, all the target bacterial strains, namely the pathogenic bacterial strains, have a positive interaction with at least one of the isolated phages. Thus, these 9 isolates provide a full coverage of target bacterial strains. The non-pathogenic strains of Pseudomonas were not affected by these phage isolates.

### Example 3

Efficacy of Bacteriophage Treatment in-Planta

The efficacy of treatment with a combination of bacteriophages in controlling symptoms of spot and speck diseases caused by Xanthomonas campestris p.v. vesicatoria (Xcv) and Pseudomonas syringae p.v. tomato (Pst), respectively, was evaluated in a field of tomato plants affected by these two bacteria strains.

The bacteriophage cocktail contained a combination of the following bacteriophages: EP007, EP017, EP022, EP026, EP027, and EP029.

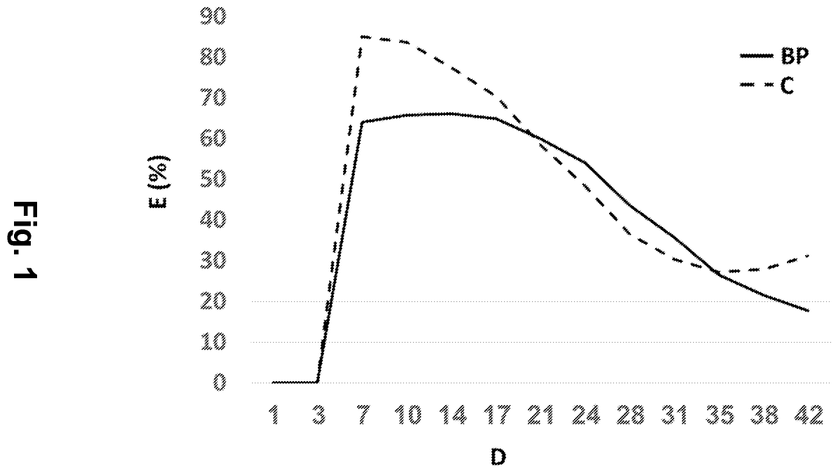

As shown in FIG. 1, consecutive treatments of the bacteriophage combination resulted in over 50% efficacy in controlling Xcv and Pst. The efficacy in controlling Xcv and Pst infection of the bacteriophage treatment was comparable to the efficacy of treatment with the known copper-based bactericide, Cupravit® Hydro.

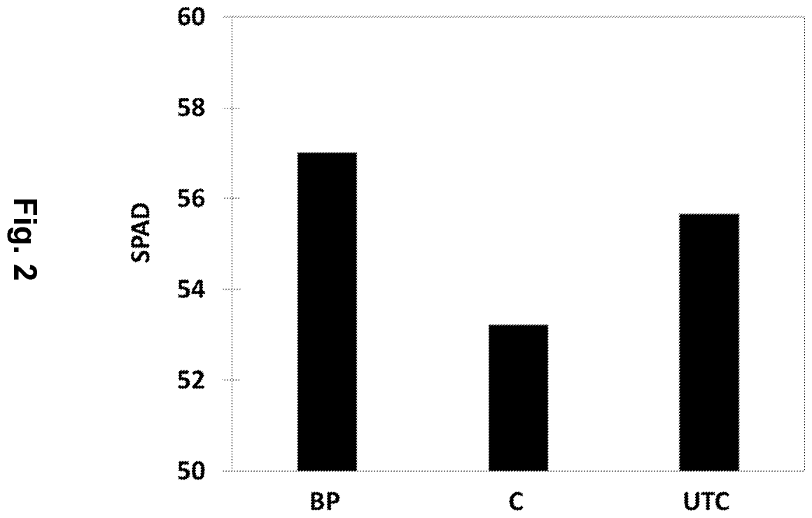

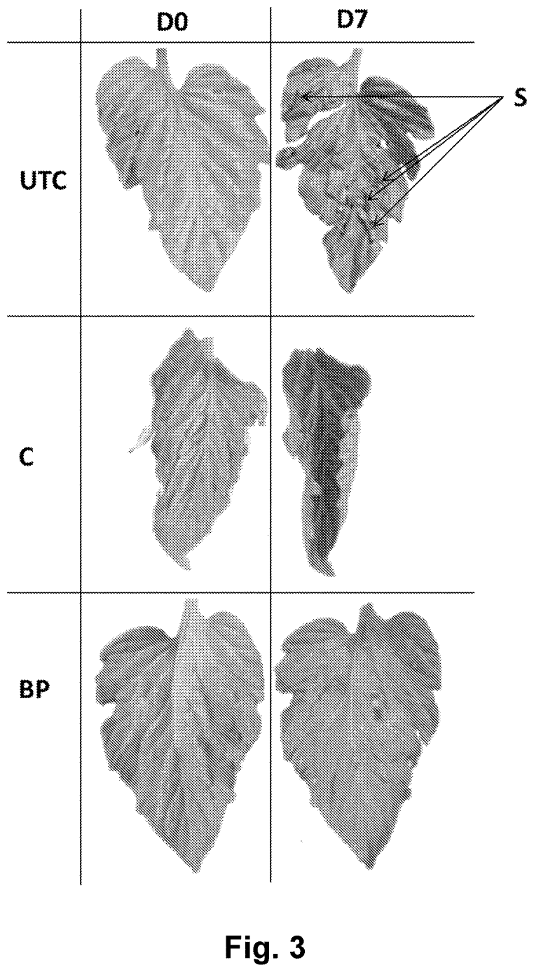

It was also observed that the treatment with the bacteriophage cocktail led to a statistically significant increase in chlorophyll concentration in the leaves of the treated plants compared to untreated plants or plants treated with Cupravit® Hydrot (FIG. 2). Additionally, the leaves of plants treated with the bacteriophage cocktail had an overall healthier appearance after 7 days from the first application of the treatment than the leaves of untreated plants or leaves of plants treated with Cupravit® Hydrot (FIG. 3). These results demonstrate the superiority of bacteriophage treatment over known copper-based bactericides that may be helpful in treating bacterial infection, but have other toxic effects of plants.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

It is the intent of the applicant(s) that all publications, patents, and patent applications referred to in this specification are to be incorporated in their entirety by reference into the specification, as if each individual publication, patent, or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-40. (canceled)

41. An agricultural composition comprising:
(i) at least one isolated bacteriophage capable of infecting a plant pathogen of the genus Xanthomonas, said at least one bacteriophage having a genomic nucleic acid sequence at least 85% identical to the nucleic acid sequence as set forth in SEQ ID NOs: 5, 6, 7, 8, or 9; and
(ii) an agriculturally acceptable carrier,
wherein the composition comprises no more than 10 different strains of bacteriophage.

42. The agricultural composition of claim 41, wherein the composition comprises at least two bacteriophages capable of infecting the pathogen of the genus Xanthomonas, wherein the composition comprises:
A:
(i) a first bacteriophage having a genomic nucleic acid sequence at least 85% identical to the nucleic acid sequence as set forth in SEQ ID NO: 5; and
(ii) a second bacteriophage having a genomic nucleic acid sequence at least 85% identical to the nucleic acid sequence as set forth in SEQ ID NO: 6;
or
B:
(i) a first bacteriophage having a genomic nucleic acid sequence at least 85% identical to the nucleic acid sequence as set forth in SEQ ID NO: 5; and
(ii) a second bacteriophage having a genomic nucleic acid sequence at least 99% identical to the nucleic acid sequence as set forth in SEQ ID NO: 9.

43. The agricultural composition of claim 41, further comprising at least one additional isolated bacteriophage capable of infecting a plant pathogen of the species Pseudomonas syringae pv. tomato, wherein said at least one additional bacteriophage has a genomic nucleic acid sequence at least 85% identical to the nucleic acid sequence as set forth in SEQ ID NOs: 1, 2, 3, or 4.

44. The agricultural composition of claim 43, wherein said at least one bacteriophage capable of infecting Pseudomonas syringae pv. tomato comprises at least three non-identical bacteriophages capable of infecting Pseudomonas syringae pv. tomato, wherein said at least three non-identical bacteriophages capable of infecting Pseudomonas syringae pv. tomato comprise:
A:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2; and
(iii) a bacteriophage having a genomic nucleic acid sequence at least 85% identical to the nucleic acid sequence as set forth in SEQ ID NO: 3;
or
B:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2; and
(iii) a bacteriophage having a genomic nucleic acid sequence at least 85% identical to the nucleic acid sequence as set forth in SEQ ID NO: 4.

45. The agricultural composition of claim 43, comprising:
A:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 5; and
(iv) a bacteriophage having a genomic nucleic acid sequence at least 85% identical to the nucleic acid sequence as set forth in SEQ ID NO: 6;
or
B:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
C:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
D:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
E:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
F:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 4;
(v) a bacteriophage having a genomic nucleic acid sequence at least 85% identical to the nucleic acid sequence as set forth in SEQ ID NO: 5;
(vi) a bacteriophage having a genomic nucleic acid sequence at least 85% identical to the nucleic acid sequence as set forth in SEQ ID NO: 7;
(vii) a bacteriophage having a genomic nucleic acid sequence at least 85% identical to the nucleic acid sequence as set forth in SEQ ID NO: 8; and
(viii) a bacteriophage having a genomic nucleic acid sequence at least 85% identical to the nucleic acid sequence as set forth in SEQ ID NO: 9.

46. The agricultural composition of claim 41, wherein the at least one isolated bacteriophage capable of infecting a plant pathogen of the genus Xanthomonas is a bacteriophage deposited under accession number F/00201, F/00202, F/00203, or I-6020.

47. The agricultural composition of claim 43, wherein the at least one isolated bacteriophage capable of infecting a plant pathogen of the species Pseudomonas syringae pv. tomato is a bacteriophage deposited under accession number F/00196, F/00197, F/00198, or F/00200.

48. The agricultural composition of claim 41, further comprising an agent which promotes the growth of a plant or maintains the health of the plant, wherein said agent is selected from the group consisting of a fertilizer, an acaricide, a heribicide, a fungicide, an insecticide, a nematicide, a pesticide, a plant growth regulator, a rodenticide, and a nutrient.

49. The agricultural composition of claim 41, wherein said agriculturally acceptable carrier comprises at least one agent selected from the group consisting of a stabilizer, a tackifier, a preservative, a surfactant, an adjuvant, a salt, and any combination thereof.

50. An article of manufacture comprising:
(i) at least one bacteriophage capable of infecting a plant pathogen Pseudomonas syringae pv. Tomato, the bacteriophage having a genomic nucleic acid sequence at least 85% identical to the nucleic acid sequence as set forth in SEQ ID NOs: 1, 2, 3, or 4; and
(ii) at least one bacteriophage capable of infecting a plant pathogen of the genus Xanthomonas having a genomic nucleic acid sequence at least 85% identical to the nucleic acid sequence as set forth in SEQ ID NOs: 5, 6, 7, 8, or 9;
wherein the composition comprises no more than 10 bacteriophages capable of infecting pathogens of the species Pseudomonas syringae pv. tomato and pathogens of the genus Xanthomonas.

51. The article of manufacture of claim 50, wherein said at least one bacteriophage capable of infecting the pathogen Pseudomonas syringae pv. tomato and said at least one bacteriophage capable of infecting the pathogen of the genus Xanthomonas are formulated in a single composition or in separate compositions.

52. The article of manufacture of claim 50, comprising:
A:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 5; and
(iv) a bacteriophage having a genomic nucleic acid sequence at least 85% identical to the nucleic acid sequence as set forth in SEQ ID NO: 6;
or
B:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
C:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 9;
or
D:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
E:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 4;
(iv) a bacteriophage having a genomic nucleic acid sequence at least 85% identical to the nucleic acid sequence as set forth in SEQ ID NO: 5;
(v) a bacteriophage having a genomic nucleic acid sequence at least 85% identical to the nucleic acid sequence as set forth in SEQ ID NO: 7; and
(vi) a bacteriophage having a genomic nucleic acid sequence at least 85% identical to the nucleic acid sequence as set forth in SEQ ID NO: 8;
or
F:
(i) a bacteriophage having a genomic nucleic acid sequence at least 85% identical to the nucleic acid sequence as set forth in SEQ ID NO: 1;
(ii) a bacteriophage having a genomic nucleic acid sequence at least 85% identical to the nucleic acid sequence as set forth in SEQ ID NO: 2;
(iii) a bacteriophage having a genomic nucleic acid sequence at least 85% identical to the nucleic acid sequence as set forth in SEQ ID NO: 3;
(iv) a bacteriophage having a genomic nucleic acid sequence at least 85% identical to the nucleic acid sequence as set forth in SEQ ID NO: 4;
(v) a bacteriophage having a genomic nucleic acid sequence at least 85% identical to the nucleic acid sequence as set forth in SEQ ID NO: 5;
(vi) a bacteriophage having a genomic nucleic acid sequence at least 85% identical to the nucleic acid sequence as set forth in SEQ ID NO: 7;
(vii) a bacteriophage having a genomic nucleic acid sequence at least 85% identical to the nucleic acid sequence as set forth in SEQ ID NO: 8; and
(viii) a bacteriophage having a genomic nucleic acid sequence at least 85% identical to the nucleic acid sequence as set forth in SEQ ID NO: 9.

53. The article of manufacture of claim 50, wherein the at least one isolated bacteriophage capable of infecting a plant pathogen of the genus Xanthomonas is a bacteriophage deposited under accession number F/00201, F/00202, F/00203, or I-6020.

54. The article of manufacture of claim 50, wherein the at least one isolated bacteriophage capable of infecting a plant pathogen of the species Pseudomonas syringae pv. tomato is a bacteriophage deposited under accession number F/00196, F/00197, F/00198, or F/00200.

55. A method of controlling or preventing bacterial spot disease of a tomato or pepper plant comprising contacting the tomato or pepper plant with an effective amount of at least one bacteriophage capable of infecting a pathogen of the genus Xanthomonas, said at least one bacteriophage having a genomic nucleic acid sequence at least 85% identical to the nucleic acid sequence as set forth in SEQ ID NOs 5, 6, 7, 8, or 9, thereby controlling or preventing the bacterial spot disease of the tomato or pepper plant.

56. The method of claim 55, wherein the at least one bacteriophage capable of infecting a pathogen of the genus Xanthomonas, is a bacteriophage having a genomic nucleic acid sequence at least 85% identical to the nucleic acid sequence as set forth in SEQ ID NOs: 5 or 6, or a bacteriophage having a genomic nucleic acid sequence at least 99% identical to the nucleic acid sequence as set forth in SEQ ID NOs: 7, 8 or 9.

57. A method of controlling or preventing bacterial spot disease and bacterial speck disease of a tomato plant comprising contacting the tomato plant with an effective amount of the agricultural composition of claim 41, thereby controlling or preventing the bacterial spot disease and bacterial speck disease of the tomato plant.

58. The method of claim 55, wherein said contacting is carried out in the vicinity of or onto: a shoot, root, stalk, stem, trunk, seed, fruit, flower, leaf, or any combination thereof.

59. The method of claim 55, wherein said contacting comprises dipping, spraying, irrigating, drenching, or fogging.

60. The method of claim 55, wherein the contacting is repeated daily, weekly, twice a week, every other week, or monthly.
